(19) United States
(12) Patent Application Publication
(10) Pub. No.: US 2011/0250256 A1
(43) Pub. Date: Oct. 13, 2011

(54) Dissolvable Nano Web Porous Film and Method of Preparing the Same

(75) Inventors: Yoo Hyun-Oh, Seoul (KR); Son Tae-Won, Daegu (KR)

(73) Assignee: GENIC CO, LTD., Seoul (KR)

(21) Appl. No.: 12/672,950

(22) PCT Filed: Sep. 7, 2007
(86) PCT No.: PCT/KR2007/004342

§ 371 Date: Jun. 28, 2011

(30) Foreign Application Priority Data
Aug. 10, 2007 (KR) 10-2007-0080677

(51) Int. Cl. A61K 9/00; A61K 31/135; B29C 47/00; A61K 31/445; A61K 31/4439; A61K 31/4748; A61K 31/44

(52) U.S. Cl. 424/439; 424/400; 514/289; 514/653; 514/357; 514/327; 514/343; 264/465

(57) ABSTRACT

A dissolvable nano web porous film has a network structure and a large specific surface area. The network structure is formed by stacking nano fibers that have a thickness of 10˜50,000 nm. The nano fibers are formed of a composition comprising nano fiber forming polymer, a gelling agent, a plasticizer, an optional active component and an optional additive. A method of preparing a dissolvable nano web film comprises producing a polymer melt or solution formed of a composition that comprises a nano fiber forming polymer, a gelling agent, a plasticizer, an optional active component and additives, spinning the melt or solution to form a nano web porous film having nano webs stacked in a network structure, drying the nano web porous film, and stabilizing the nano web porous film. The porous film has a 5˜5000 times higher specific surface area and a 2˜20 times faster dissolving time than a conventional film. The dissolvable nano web porous film has excellent dissolvability and solubility.

### TECHNICAL FIELD

The present invention relates to a dissolvable nano web porous film that is biologically or physiologically applicable, and a method of preparing the same. More particularly, the present invention relates to a nano web porous film that has a nano web network structure and a large specific surface area and a method of preparing the same.

### BACKGROUND ART

Generally, conventional edible nonporous films are produced in such a fashion of:

dissolving polymers such as starch, pullulan, etc., and materials which include film forming agents, plasticizer, flavoring agents, sulfur precipitating agents, saliva stimulating agents, cooling agents, surfactants, stabilizing agents, emulsifying agents, thickening agents, binding agents, coloring agents, pigments, sweeteners, fragrances, antimicrobial agents, etc. in a solvent such as water to provide a mixture thereof; forming the mixture into a film, followed by drying the film; cutting the obtained film to pieces of the film having a proper size; and putting them in a container.

The conventional edible nonporous films require excellent dissolvability in a mouth such that the films are easily liquefied in the mouth, and drying resistance whereby the films are not cracked or curled. Further, the films should not become sticky due to absorption of moisture or heat.

Industrially, natural starch is used as a main ingredient for prancing a foal packaging film. However, as the starch has high viscosity, it is necessary to decrease the concentration of starch to form an edible thin nonporous film.

Such a thin nonporous film tends to absorb moisture, which causes sticking of the film. In the case of containing protein such as gelatin as a main ingredient, the film tends to absorb moisture or tends to be dissolved at high temperature and humidity, and thus it can easily become sticky in the summer.

JP Patent Publication No. 1995-100186, JP Patent Publication No. 1993-220203, and JP Patent Publication No. 1999-116469 disclose a film capable of being quickly dissolved in the mouth. JP Patent Publication No. 1993-236885 discloses an edible film as a vehicle to carry flavoring and refreshing foal without attracting public attention, and a film that contains pullulan and carrageenan to improve drying resistance. However, these films have low resistance to moisture and suffer from sticking in highly humid conditions.

U.S. Pat. No. 3,784,390 discloses pullulan films and their use in coating and packing materials for foods, pharmaceuticals and other oxygen sensitive materials. U.S. Pat. No. 4,623,394 discloses a methcd of preparing a film with pullulan. U.S. Pat. No. 7,067,116 discloses products of edible films which include pullulan binders, polysaccharides, antibacterial agents, flavor-imparting agents, and pharmaceutically active components.

U.S. Pat. No. 6,528,088 discloses a film that contains modified starch, such as etherified starch or esterified starch, and plasticizers mixed with the starch in the ratio of at least 0.5:1 by weight to increase the plasticity of the film. However, a high plasticizer content of the film causes an unpleasant feeling in the mouth and insufficient sticking resistance.

U.S. Pat. No. 6,656,493 discloses a film that contains sodium alginate and maltodextrin. Maltodextrin having a low molecular weight is added to the films to improve dissolution in the oral cavity. However, as the concentration of maltcdextrin increases, moisture resistance of the film decreases, causing easy sticking thereof. The drying resistance of the films is also not satisfactory.

US Patent Application Publication No. 2003/0099692 discloses a film that includes processed starch as a main ingredient. For the thin film, the starch is cross-linked and chemically processed as in acetyl, ester, hydroxyethyl and hyiroxyproply derivatives of starch. Further, derivatives of starch by oxidation, enzyme conversion, acid hydrolysis, etc. may also be used in forming the film. The processed starch is substantially independently used, and thus the film does not have sufficient sticking resistance.

Conventional methods relating to administration of a pharmaceutical active agent in an edible film vehicle are disclosed in the following documents: WO 98/26763 discloses a film for use in the oral cavity, which may contain a cosmetic or pharmaceutical active component; WO 01/70194 discloses a rapidly dissolvable edible film that contains an ion exchange resin as a taste masking agent; WO 98/20862 also discloses a film for use in the oral cavity, which may contain a cosmetic or pharmaceutical active component; WO 99/17753 discloses a rapidly soluble film for delivering medical supplies to be absorbed in a digestive tract; and U.S. Pat. No. 6,596,290 discloses the delivery of pharmaceutical agents in an edible film vehicle.

Meanwhile, there are methuls including spun-lacing, spun-bonding, melt-blown, flash spinning, electro spinning, etc. to successively produce webs directly from a polymer solution. Since these methods are applicable to a variety of polymers and can produce polymer webs instantly in a liquid condition, the polymer webs can be formed by these very simplified processes as compared to conventional spinning methods. Recently, these techniques are applied to formation of an electrolyte layer, an isolation layer, a filter, a dressing web, a barrier web, a scaffolder, a sensor web, etc. based on a polymer web of minute and superfine fibers.

U.S. Pat. No. 5,935,883 discloses a process of producing a superfine microfiber split web using a melt-blown device. U.S. Pat. No. 6,057,256 discloses formation of a coherent entangled web by blowing a polymer solution in a collector. U.S. Pat. No. 6,106,913 discloses a web produced with a fiber of 1 nm using the combination of an electro spinning methcd and an air vortex spinning methcd. U.S. Pat. No. 6,110,509 discloses a biodegradable silk web having a diameter ranging from 2 to 2000 nm using an electro spinning methcd.

KR patent publication No. 2001-0031586 discloses a process of massively preparing a nano-sized nanofiber web by organically combining a melt-blown methcd and an electrostatic spinning methcd. KR patent publication No. 2001-0031587 discloses a process of preparing a nanofiber web by organically combining a flash spinning methcd and an electrostatic spinning methal. KR patent publications Nos. 2003-0077384 and 2004-0040692 disclose a method of producing a superfine nanofiber web which is improved in productivity and yield by combination of the melt-blown spinning methcd and the electrostatic spinning methal.

As described above, a variety of patents and applications disclose the methcd of preparing the edible nonporous film. However, there is still a need for an edible web porous film having excellent dissolvability, drying resistance, and sticking resistance, and such a need can be satisfied by the present invention that provides an edible web porous film having a nano-network structure, as described below.

### DISCLOSURE OF INVENTION

Technical Problem

The present invention has been male to solve the foregoing problems of the prior art and therefore an aspect of the present invention is to provide a dissolvable nano web porous film that is biologically and physiologically applicable.

Another aspect of the invention is to provide a dissolvable nano web porous film that is quickly dissolved in saliva in administration into the oval cavity or fluid secreted from the skin.

A further aspect of the present invention is to provide an edible nano web porous film, of which active component is absorbed into the body and which is edible without water.

Yet another aspect of the present invention is to provide a nano web porous film that has superior dissolvability, drying resistance, and sticking resistance.

Yet another aspect of the present invention is to provide a dissolvable nano web porous film where an active component and the film are capable of being rapidly decomposed or dissolved.

Yet another aspect of the present invention is to provide a method of preparing a dissolvable nano web porous film having the foregping properties.

Technical Solution

According to an aspect of the invention, the present invention provides a dissolvable nano web porous film having a network structure, wherein the network structure is formed by stacking nano fibers, the nano fibers having a thickness of 10˜50,000 nm and formed of a composition composed of (A) a nano fiber forming polymer, (B) a gelling agent, and (C) a plasticizer.

According to another aspect of the invention, the present invention provides a method of preparing a dissolvable nano web film, comprising: (1) forming a polymer melt or solution of a composition composed of (A) a nano fiber forming polymer, (B) a gelling agent and (C) a plasticizer, as essential ingredients, and (D) active components, and (E) additives, as optional ingredients; (2) spinning the melt or solution to form a nano web porous film having nano webs stacked in a three-dimensional network structure; (3) drying the nano web porous film; and (4) stabilizing the nano web porous film.

Advantageous Effects

As set forth above, according to the present invention, the dissolvable nano web porous film has a 5˜5000 times lager specific surface area and a 2˜20 times faster dissolving time than a conventional film. Thus, the dissolvable nano web porous film is excellent in dissolvability and solubility.

According to the present invention, the dissolvable nano porous film is used for a medicine to be edible without water. In this case, since the film is rapidly dissolvable and liquefied by saliva in the oral cavity, the medicine is absorbed into the blocdstream through a mucous membrane in the oral cavity without any displeasure. As such, since the medicine is absorbed into the bloodstream without passing through the stomach and gullet, it is advantageous in rapidly providing therapeutic effects.

The dissolvable nano porous film according to the present invention can be preferably used as a carrier for functional foals, drugs, plant extracts, etc. which have a very small quantity of effective ingredients.

According to the present invention, the dissolvable nano web porous film is provided along with emulsified fat-soluble flavors and emulsified oils and fats to be used as a mouth-freshening nano web porous film or a flavor nano web porous film for bad breath prevention, refreshment, improvement of edibility, nutrients supplementation, drug delivery, improvement of health, etc. As a result, these films can be a substitute for gum, candies, a spray type oral cleansing agent, a breath freshening agent, and the like.

According to the present invention, since the dissolvable nano web porous film is dissolved by fluid secreted from the skin or moisture to be rapidly soluble and liquefied, medicines or functional materials are absorbed epidermally and percutaneously without displeasure. Accordingly, therapeutic effects are quickly exhibited, and thus the film can be useful for skin care, therapy for skin, etc.

According to the present invention, the dissolvable nano web porous film is provided along with a bleaching agent or a dental bleaching agent to be used as a dental bleaching film.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a mimetic diagram of a portion of a network structure on the surface of a dissolvable nano web porous film according to an exemplary embodiment of the present invention, in which reference numeral 1 indicates a cross section of a nano fiber in the dissolvable nano web porous film, and reference numeral 2 indicates a pore between the nano fibers;

FIG. 2 is a micrograph of the surface of the nano web porous film taken by a scanning electron microscope (SEM) according to a first exemplary embodiment of the present invention, illustrating that plural nano fibers are stacked to form a nano web where a network structure and pores exist;

FIG. 3 is an enlarged SEM image of a portion of the surface of the dissolvable nano web porous film according to the first exemplary embodiment of the present invention;

FIG. 4 is a highly enlarged SEM image of a portion of the surface of the dissolvable nano web porous film according to the first exemplary embodiment of the present invention;

FIG. 5 is an SEM image of the cross section of the dissolvable nano web porous film according to the first exemplary embodiment of the present invention;

FIG. 6 is an SEM image of the surface of a nonporous film according to a second exemplary embodiment of the present invention;

FIG. 7 is an SEM image of the cross section of the nonporous film according to the second exemplary embodiment of the present invention; and

FIG. 8 is a graph illustrating the relation (i.e., dissolvability in tepid water and in the oral cavity) between the dissolving time and the thickness of the dissolvable nano web porous film according to the first exemplary embodiment of the present invention and the relation between the dissolving time and the thickness of the nonporous film according to the second exemplary embodiment of the present invention.

### BEST MODE FOR CARRYING OUT THE INVENTION

Exemplary embodiments of the present invention will now be described in detail with reference to the accompanying drawings.

The present invention relates to a dissolvable nano web porous film that can be rapidly dissolved by saliva in the oral cavity or fluid secreted from the skin, and a method of preparing the same. Unlike a conventional nonporous film, the nano web porous film has a large surface area, pores and a rapidly soluble network structure capable of rapidly responding to materials from the outside, and is rapidly dissolved by the saliva in the oral cavity or the fluid secreted from the skin. Due to the film's rapid dissolvability, active components (e.g., drugs and the like) in the film can be more effectively absorbed into the body. Also, the film has edibility to allow the film to be edible without water and without any displeasure in a dissolved state, dissolvability, drying resistance, and sticking resistance.

The dissolvable nano web porous film according to the exemplary embodiment of the present invention has a network structure where nano fibers having a thickness of 10˜50,000 nm are stacked. Here, the nano fibers are formed of a composition composed of (A) a nano fiber forming polymer, (B) a gelling agent, and (C) a plasticizer. If the nano fiber has a thickness less than 10 nm, it is difficult to mechanically produce the film. If the nano fiber has a thickness more than 50,000 nm, the film has reduced dissolvability.

The nano web porous film is preferably formed to have a thickness of 5˜500 μm to obtain excellent properties in view of drying resistance, feeling in the oral cavity, dissolvability in the oral cavity, etc. The nano web porous film preferably has porosity ranging from 10˜90%. If the porosity is less than 10%, it is difficult to mechanically produce the film. If the porosity is more than 90%, it is difficult to form a nano web. The nano web porous film preferably has a specific surface area in the range of 1˜1,000 m²/g. If the specific surface area is less than 1 m²/g, the film may not exhibit excellent dissolvability. If the specific surface area is more than 1,000 m²/g, it is difficult to mechanically produce the film.

Hereinafter, a composition and a method for forming the film according to the present invention will be described in detail.

1. Composition for Nano Web Porous Film

According to the present invention, the dissolvable nano web porous film (hereinafter, also referred to as a “porous film”) is formed of nano fibers having a composition that essentially consist of (A) a nano fiber forming polymer, (B) a gelling agent, and (C) a plasticizer. Optionally, the composition, which forms the nano fiber, may further comprise (D) an active component andbr (E) other ailitives according to use of the porous film, if necessary.

(A) Nano Fiber Forming Polymer

According to the present invention, the nano fiber forming polymer (A), an ingredient of the composition for forming the (dissolvable andbr edible) nano web porous film, is a main ingredient for the nano fiber and the nano web porous film, and carries active components in a nano web.

Examples of the nano fiber forming polymer (A) used to form the film of the invention include, but are not limited to, pullulan, hydroxypropylmethyl cellulose (HPMC), h)droxymethyl cellulose (HMC), hydroxyethyl cellulose (HEC), hydroxypropyl cellulose (HPC), ethyl cellulose (EC), methyl cellulose (MC), hydroxypropylmethyl cellulose phthalate (HPMCP), hydroxypropylmethyl cellulose acetate succinate (HPMCAS), cellulose acetate phthalate (CAP), carboxymethyl cellulose (CMC), polyvinyl pyrrolicbne (PVP), polyvinyl alcohol (PVA), sodium alginate, polyethylene glycol, locust bean gum, xanthan gum, tragacanth gum, guar gum, acacia gum, arabic gum, polyacrylic acid, methyl methacrylate copolymer, amylose, dextrin, chitin, chitosan, levan, elsinan, collagen, gelatin, glucan, high amylose starch (corn starch, potato starch, wheat starch, rice starch, mung-bean starch, yellow bean starch, etc.), ester derivatives of starch, soy protein isolate, whey protein isolate, casein, gellan, pectin, and a combination thereof. Also, a mixture of two or more aforementioned materials may be used as the nano fiber forming polymer (A). The nano fiber forming polymer (A) has an average molecular weight of 1,000˜2,000,000 g/mol. If the average molecular weight is less than 1,000 g/mol, the viscosity of the polymer is low, making it difficult to form a nano web. If the average molecular weight is more than 2,000,000 g/mol, the viscosity is high, making it difficult to process the polymer. In addition to the aforementioned polymers, any of known hydrophilic natural polymers having an average molecular weight of 1,000˜2,000,000 g/mol may be used to form the film of the invention.

The nano fiber forming polymer (A) is added in an amount of 1˜99 wt %, and preferably, of about 15˜80 wt %, to the composition of the nano web porous film. If the polymer content is less than 1 wt %, it is difficult to form a fiber. And, since other solvents or ingredients should also be mixed in formation of the nano fiber, the polymer content may be aided up to 99 wt %.

The other ingredients (specifically, the gelling agent, plasticizer, active ingredients, additives, and water) of the composition are added to the composition according to proposed contents and conditions, which will be described below.

(B) Gelling Agent

The gelling agent, an ingredient of the composition to form the dissolvable nano web porous film, is used to prevent the nano web porous film from breaking, and becomes gel when cooled. Examples of the gelling agent include, but are not limited to, a natural gelling agent, such as locust bean gum, xanthan gum, carrageenan, gelatin, gellan gum, agar, alginates, guar gum, starch, pectin, ethyl cellulose, methyl cellulose, scdium carboxymethyl cellulose, polyethylene glycol, and combinations thereof. Also, a mixture of two or more aforementioned materials may be used as the gelling agent.

A mixing ratio of the gelling agent (B) to 100 wt % polymer (A) is in the range of about 0.1˜50 wt % (for example, 0.1˜50 parts by weight gelling agent to 100 parts by weight polymer), and preferably, of 0.4˜30 wt %. If the mixing ratio is below the range, the film tends to be curled. Conversely, if the ratio exceeds the range, the film has low dissolvability.

(C) Plasticizer

The plasticizer is used to endow the film with properties of hardening prevention, sticking prevention, and plasticity. The plasticizer also drops the glass transition temperature of the film during the process, thereby adjusting the melting viscosity thereof. Examples of the plasticizer includes, but are not limited to, sorbitol, mannitol, glycerin, propylene glycol, polyethylene glycol, fatty acid glycerol monoester, sugar alcohol, monosaccharide, oligosaccharide, triethylene glycol, myvacet, triethyl citrate, triacetin, propylene glycol monocaprylate, propylene glycol dicaprylate, miglyol, etc., all of which are organic compounds of polyol containing at least one hydroxyl group. Further, alcohol-based or polyol-based organic compounds incluing at least one hydroxyl group may be used as the plasticizer. These materials are used independently or together. A mixture of two or more aforementioned materials may be used as the plasticizer. A mixing ratio of the plasticizer (C) to 100 wt % polymer (A) is in the range of about 0.1˜40 wt % (for example, about 0.1˜40 parts by weight plasticizer to 100 parts by weight polymer). If the mixing ratio is below the range, the film deteriorates in drying resistance. If the ratio is above the range, the film deteriorates in sticking resistance.

(D) Active Components

Pursuant to use of the dissolvable nano web porous film, the composition may further comprise an active component as necessary. The active component comprises food, medicines, drugs, functional materials, etc., which promote structural and functional modifications in the body to which it is administered.

The active component is not specifically limited, but provided as any of materials which are physiologically applicable, mixable with other materials of the nano web porous film, and have activity corresponding to uses of the film. Examples of the active component include, but are not limited to, one selected from the group or type consisting of: α-adrenergic agonists; β-adrenergic agonists; α-adrenergic blocking agents; β-adrenergic blocking agents; alcohol prohibition agents; aldose-reductase inhibitors; anabolic agents; narcotic analgesics; derivatives of morphine; non-narcotic analgesics, preferably, salicylates and derivatives thereof; androgens; anesthetics; appetite inhibitors; anthelmintics (taenia, nematode, filarial, schistosome, and fluke eliminating active agents); antiacne agents; antiallergic agents; antiamebic drugs (ameba eliminating agents); antiandrogens; angina therapeutic agents; antiarrhythmic drugs; antiarteriosclerotic agents; antiarthritic and antirheumatic agents; antimicrobial agents (antibiotics), preferably, aminoglycosides, amphenicols, ansamycins, β-lactams (particularly, carbapenems, cephalosporins, cephamycins, monolactams, oxacephems, and penicillins), lincosamiles, macrolides, polypeptides and tetracyclines; synthetic antimicrobials, preferably, 2,4-diaminopyrimidines, nitrofurans, quinolones and quinolone analogues, sulfonamides and sulfones; anticholinergics; anticonvulsants; antidepressants, preferably, bicyclic antidepressants, hydrazides, hydrazines, pyrrolidones, tetracycline antidepressants; tricyclic antidepressants, polycyclic imides; antidiabetic agents, preferably, biguanides, sulfonylurea derivatives; antidiarrheals; antidiuretic agents; antiestrogens; antifungal agents/fungicides, preferably, polyenes; synthetic antifungal agent/fungicide, preferably, allyl amines, imidazoles, triazoles; antiglaucomatous agents; antigpnado-trophins; antiguut agents; antihistamines, preferably, alkylamine derivatives, aminoalkyl ethers, ethylenediamine derivatives, piperazines, tricyclic compounds (particularly, phenothiazines); antihyperlipoproteinemia agents (fat inhibitors), preferably, aryloxy alkane acid derivatives (particularly, clofibric acid derivatives and analogues), bile acid-masking materials, HMG-CoA-reciactase inhibitors, nicotinic acid derivatives, thyroid hormones and analogues thereof; antihypertensive drugs/hypotensives, preferably, benzothiadiazine derivatives, N-carboxylalkyl-(peptideflactam) derivatives, guanidine derivatives, hydrazines/phthalazines, imidazole derivatives, quaternary ammonium compounds, quinazoline derivatives, reserpine derivatives, sulfonamide derivatives; antihyperthyroid agents; antihypotensive agents; antihypothyroid agent; non-steroidal antiinflammatory drugs (antiphlogistics), preferably, aminoarylcarboxyl acid, arylacetic acid, arylbutyric acid, arylcaboxylic acid and arylpropionic acid derivatives, pyrazoles, pyrazolones, salicylic acid derivatives, thiazine carboxamide; antimalarial drugs, preferably, quinines, salts thereof, acids thereof and derivatives thereof; antimigraine drugs; retching inhibitors; antineoplastic drugs, preferably, alkylating drugs (particularly, alkyl sulfonates, aziridines, ethylenimines, methyl melamines, nitrogen mustard gases, and nitrosoureas); antibiotics, antimetabolic agents (particularly, folinic acid analogues, purine analogues and pyrimidine analogues), enzymes, interferons, interleukins; hormonal antineoplastics, preferably, androgens, antiairenalines, antiandrogens, antiestrogens (particularly, aromatase inhibitors); antineoplastic food adJitives; antiparkinsonian agents; antipheochromocytoma agents; antipneumocystis agents; prostatic hypertrophy therapeutic agents; protozoacides, preferably, antileishmania, antitrichomonal and anti-trypanosoma drugs; antipruriginous agents; antipsoriasis drugs; antineuroleptic agents, preferably, butyrophenones, phenothiazines, thioxanthenes, tricyclic drugs, 4-arylpiperazines, 4-arylpiperidines; antipyretics; antirickettsia agents; antiseborrheic agents; antiseptics, preferably, guanidines, halogens and halogen compounds, nitrofuran, phenols, quinolines; antiseizur drugs, anti-convulsants/convulsion inhibitors; antithrombotics; antitussives; antiulcer drugs; uric acid eliminating agents (antiurolithiasis agents); antiophidicas; antiviral drugs, preferably, purines, pyrimidinones; anxiolytic, preferably, arylpiperazines, benzodiazepine derivatives, carbamates; benzodiazepine antagonists; bronchodilators, preferably, ephedrine derivatives, quaternary ammonium compounds, xanthine derivatives; calcium channel blockers, preferably, arylalkylamines, dihydropyridine derivatives, piperazine derivatives; calcium modulators; cardiac stimulants; chelate or complex forming agents; cholecystokinin antagonists; antigallstone agents; bile secretagugues; parasympathomimetic drugs; cholinesterase inhibitors; cholinesterase rehabilitation materials; CNS stimulants; decongestants; tooth decay prevention agents; decolorants; diuretics, preferably, organomercury compounds, pteridines, purines, steroids, sulfonamides, uracils; dopamine receptor agonists; ectoparasite eliminating agents; enzymes, preferably, digestive enzymes, penicillin inactivating enzymes, proteolytic enzymes; enzyme inducers; steroidal and non-steroidal estrogens; gastric acid secretion inhibitors; glucocorticoids; gonadotropic activators; gonaiotropin hormones; growth hormone inhibitors; growth hormone releasing factors; growth stimulants; anticoagulants; heparin antagonists; liver protective agents; liver disease therapeutic agents; immune modulators; immune inhibitors; ion exchange resins; prolactins; LH-RH agpnists; lipotropic agents; lupus erythematosus eliminating agents; mineralocorticoids; miotic agents; monoamine oxidase inhibitors; mucolytic agents; muscle relaxants; narcotic antagonists; neuroprotective agents; nootropics; ophthalmic agents; ovarian hormones; oxytocic agents; pepsin inhibitors; peristaltic stimulants; progestogens; prolactin inhibitors; prostaglandins and analogues thereof; protease inhibitors; respiratory stimulants; hardening agents; sedatives/somnifacients, preferably, non-cyclic ureides, alcohols, amides, barbiturates acid derivatives, benzodiazepine derivatives, bromides, carbamates, chloral derivatives, piperidinediones, quinazolone derivatives; antithrombotic agents; thyroid hormones; uricosuric agents; vasodilators (cerebrum); vasodilators (coronary arteries); vasodilators (peripheral nerve); blood vessel protective agents; vitamins, provitamins, vitamin extracts, vitamin derivatives; and trauma therapeutic agents.

The active component is an optional ingredient and selectively added according to purposes of the nano web porous film. The active component is not limited to the aforementioned materials, but may employ drugs which can be pharmaceutically activated. The active component is not specifically limited to a certain component, so long as it is capable of being dissolved in water or ethanol. A proper dosage of the active component is 0.001˜50 mg.

According to the present invention, the dissolvable nano web porous film is edible without water, and rapidly dissolved and liquefied by saliva in the oral cavity, so that drugs may be absorbed into the body without any displeasure. Even in this case, it is desirable to use an active component (drugs and the like) which does not have ill-affect on digestive organs, the mucous membrane in the oral cavity, and the like. The active component is optionally provided up to 40 wt %, and preferably, 0.01˜40 wt %, with respect to the total weight of the final film. Since it is an optional component, the active component does not have any lower limit in content, but can be added in a content of 0.01 wt % or more with respect to the final film so as to exhibit its properties by the addition. If the content of the active component exceeds 40 wt %, it is difficult to form a nano web.

(E) Other Additives

If necessary, additives may be optionally ailed to the composition for the dissolvable nano web porous film to improve properties required for the film. Examples of the additives include, but are not limited to, flavoring agents, sulfur precipitating agents, saliva stimulating agents, cooling agents, surfactants, stabilizing agents, emulsifying agents, thickening agents, binding agents, coloring agents, pigments, sweeteners, fragrances, germicides, antimicrobial agents, pharmaceutical active agents, etc.

In more detail, the additives may be provided as, but are not limited to, saccharides to improve taste (all typical saccharides, for example, but not limited to, white sucrose, maltose, fructose, galactooligpsaccharkle, galactose, fructooligpsaccharide, dextrin, ion starch syrup, malt syrup, lactose, glucose, sorbitol, mannitol, xylitol, inositol, etc.); saliva stimulating agents (e.g., but not limited to, citric, lactic, maleic, succinic, ascorbic, adipic, fumaric, and tartaric acids); sweeteners (e.g., but not limited to, monosaccharide, disaccharide, ribose, glucose, mannose, galactose, fructose, sucrose, maltose, invert sugars, corn syrup solids, glythyl retinoate, white sugars, water soluble artificial sweeteners); thickening agents (e.g., but not limited to, methyl cellulose, carboxyl methyl cellulose, etc.); masking agents (e.g., but not limited to, citric, tartaric, and fumaric acids); dyes and coloring agents (e.g., but not limited to, natural pigments, tar pigments, etc.); flavoring and refreshing agents (e.g., but not limited to, refreshing agent such as menthol, peppermint oil, peppermint and spearmint, natural or synthetic fragrances, etc.), nutrient, plant extracts, herbal components, vitamins, minerals, nitroglycerin, catechin, polyphenols, enzymes, emulsifiers, seasonings, fragrances, fats, and oil; when applied onto a skin adenosine, coenzyme Q10, vitamin E, vitamin C, fibroin, amyloglucosidase, albutin, hyaluronic acid, D/DL-panthenol, neomycin, hydrocortisone acetate, etc. These materials may be used independently or together. Also, a mixture of two or more aforementioned materials may be used as the additive.

The additives are provided up to 40 wt %, and preferably, in the range of 0.01˜40 wt %, to the total weight of the final film. The additives do not have a lower limit in content, but should be added in a content of 0.01 wt % or more with respect to the final film so as to exhibit its properties by the addition. If the content of additives exceeds 40 wt %, it is difficult to form a nano web.

The composition is melted or dissolved to form a polymer melt or polymer solution. Then, the polymer melt or solution is spun into nano fibers, and the fibers are stacked, thereby producing a nano web porous film in a network structure according to the present invention. Hereinafter, the method of preparing the nano web porous film according to the present invention will be described in detail.

2. Method of Preparing Nano Web Porous Film

(1) Preparation of Polymer Melt or Polymer Solution

A composition, which comprises (A) a nano fiber forming polymer, (B) a gelling agent, and (C) a plasticizer, as essential ingredients, and (D) an active component and/or (E) additives, as optional ingredients, is melted or dissolved to prepare a polymer melt or polymer solution. Here, (A) to (E) ingredients may be ailed regardless of order, which is adjusted by those skilled in the art in consideration of properties and characteristics thereof. A single or two or more ingredients may be melted or dissolved according to proper selection by those skilled in the art in consideration of the properties and characteristic thereof.

For example, but not limited to, a polymer solution can be produced by the following process: dissolving or dispersing (A) a nano web film forming polymer in a solvent in an amount of about 1.5˜4.5 times the total amount of the polymer at room temperature or a certain temperature, adding (B) a gelling agent and (C) a plasticizer thereto, followed by dissolving them, and dissolving and dispersing (D) an active component and (E) additives as necessary. Here, water is generally used as a solvent.

(2) Formation of Nano Web Porous Film by Spinning

The polymer melt or polymer solution is spun into nano fibers. The spinning process is performed by, for example, spun-lace, spun-bond, melt-blown, flash spinning, electro spinning, etc. The nano fibers are formed in the form of non-woven fabrics by spinning the polymer melt or polymer solution. By stacking the nano fibers in the form of the non-woven fabrics on a base or winding the nano fibers around a drum, a nano web is produced in a network structure where a great number of nano fibers are stacked. Here, the nano fibers are stacked or wound evenly such that the nano web has a uniform overall thickness. As a result, the nano web having the network structure where the plural nano fibers are stacked or wound evenly can be used as a porous film of the present invention.

In spinning, for example, but not being limited to, a solid content of the polymer melt or polymer solution is preferably adjusted to a concentration of about 15˜50 wt %. If the solid content is less than 15 wt %, it is difficult to form a nano web the to low viscosity. If the solid content is more than 50 wt %, it is difficult to form a stream of polymer melt or polymer solution using electric force due to high viscosity.

The spinning process is performed, but is not limited to, as follows. The polymer melt or polymer solution is stored in a syringe, and is pressurized at a constant pressure by means of a syringe pump. Then, the pressurized polymer melt or solution in a liquid phase is pushed out through a minute opening (an inner diameter of 0.1˜2 mm) of a nozzle tip in a spinning unit via an inlet pipe. With an electric field of 1˜300 kV, and preferably, of 15˜35 kV applied, the solvent is volatilized or congeals, and nano fibers are stacked or wound on the surface of a collector (e.g., drum) disposed at a predetermined distance, thereby forming the nano web having the network structure.

In forming the nano web of the network structure by the spinning process, it is possible to regulate the porosity of the nano web, the diameter, size and form of the nano fiber, the thickness of the nano web, etc. by controlling some factors (e.g., the diameter of the nozzle tip, the intensity of electric field, the distance between the nozzle tip and the collector, the discharging speed of the melt/solution, the number of nozzles, the discharging amount of the melt/solution, the spinning time, etc.). The nano web is spun and stacked to have a thickness of about 5˜500 μm in order that the dissolvable nano web porous film has excellent drying resistance, feeling in the oral cavity, dissolvability in the oral cavity, and other properties.

According to purposes of the porous film, a network structure nano web of nano fibers, which are formed of different kinds of compositions mixed to exhibit different functions and effects, may be stacked andbr wound to produce a multilayer film that has a variety of properties.

(2)-1 Compression of The Nano Web Fbrous Film

As described in the process (2), the nano webs formed of the nano fibers and having the network structure are stacked to form the porous film, while being adjusted in thickness, density, and porosity thereof. If the nano webs are formed desired thick and densely, the nano webs themselves may be used as the nano web porous film without compression process. Optionally, the nano webs of the network structure formed at the process (2) are compressed to a desired thickness, if necessary to produce the nano web porous film. The nano web porous film has a thickness of 5˜500 μm, and preferably, of about 10˜200 μm, to obtain excellent properties relating to drying resistance, feeling in the oral cavity, dissolvability in the oral cavity, etc.

(3) Drying

To obtain excellent properties relating to drying resistance, feeling in the oral cavity, dissolvability in the oral cavity, etc., the nano web film formed in a film type is dried by hot air at about 80˜110° C. so as to have a moisture content of 5˜20 wt %. If the drying temperature is less than 80° C., the solvent is not likely to be volatilized, and the film is not dried rapidly. If the temperature is more than 110° C., the film can be deformed. Also, if the moisture content of the film is less than 5 wt %, it can be easily broken, whereas, if the content is more than 20 wt %, the film is likely to droop and become sticky.

(4) Stabilization

The dried nano web porous film is kept in a humidity adjusted space or in a thermo-hygrostat to stabilize the moisture content, and the like. Preferably, the stabilization is performed to allow the nano web porous film to have a moisture content of about 5˜15 wt % so as to achieve optimal properties relating to feeling in the oral cavity, preservative performance, etc. If the stabilized final porous film has the content of moisture less than 5 wt %, it can be easily broken. If the moisture content of the film is more than 15 wt %, it tends to droop, and becomes sticky. The stabilization is preferably performed at a temperature of 20˜30° C. and a relative humidity of 40˜50% for 1 to 48 hours in order to achieve optimal properties of the film. It should be noted that the present invention is not limited to the aforementioned stabilization conditions. For example, the stabilization can be generally performed at a temperature of 25° C. and a relative humidity of 45% for 24 hours.

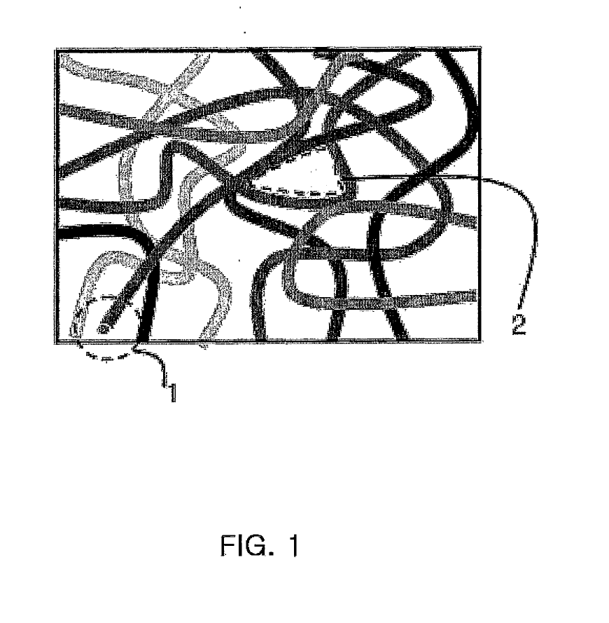

FIG. 1 is a mimetic diagram of a portion of the network structure on the surface of the dissolvable nano web porous film according to an exemplary embodiment of the invention. Reference numeral 1 indicates a cross section of a nano fiber in the dissolvable nano web porous film, and reference numeral 2 indicates a pore formed between the nano fibers. As shown in FIG. 1, according to the present invention, since the nano web porous film of the network structure comprises a plurality of nano fibers (1), which have a diameter of several to tens of thousands of nanometers and a plurality of pores (2) formed among the nano fibers, it has very high specific surface area and porosity per weight. Unlike a conventional nonporous film, since the porous film of the invention has the nano web structure which is constituted by the network structure and the pores, the porous film has excellent surface activity and dissolvability. Thus, when the porous film is taken into the oral cavity or applied to the skin, the active components (drugs and the like), carriers or other additives in the film are rapidly dissolved andior melted by saliva in the oral cavity or fluid secreted from the skin, so medicines or other active components can be more quickly discharged in the oral cavity or on the skin. More specifically, the porous film exhibits rapid dissolution with a dissolution time in the range of 1˜60 seconds, and excellent dissolvability, which is twice to twenty times higher than that of the conventional nonporous film.

For example, the dissolvable nano web porous film is decomposed and easily dissolved by reaction with amylase of saliva in the oral cavity. Thus, the dissolvable nano web porous film exhibits excellent dissolvability and rapid dissolution, particularly in the oral cavity.

Furthermore, the edible nano web porous film of the invention may be used as a package or carrier to carry functional foals, vitamins, essential minerals, drugs, etc., which can be taken through the oral cavity. Also, since flavoring agents, refined oil, etc. are ailed to the porous film when it is formed, the porous film can be used as a flavoring carrier. Thus, the film can be used in new forms for health improvement, such as bad breath prevention, refreshment, improvement of edibility, nutrients supplementation, drug delivery, health foal supplement, etc.

Since the dissolvable nano web porous film is produced including active components such as drugs, it can be formed as an edible material, which is edible without water. The porous film exhibits edibility to be rapidly dissolved and liquefied by saliva in the oral cavity, so that medicines can be absorbed in the bcdy without any displeasure. The porous film is excellent in view of dissolvability, drying resistance, and sticking resistance, which are fundamental properties required for a dissolvable film. Further, the porous film is biologically and physiologically applicable. That is, the porous film has a solubility that is physiologically allowable in the skin and mucous membranes such as the oral cavity, the gullet, etc.

On the other hand, the conventional nonporous film should have a certain thickness to perform intended functions, e.g., to load active components. Thus, the thickness of the film must be determined by the properties and amount of the active components to be discharged from the film. Generally, an increase in thickness of the film leads to a decrease in decomposition and dissolution rates of the nonporous film. In particular, not only a thin nonporous film but also a thick nonporous film tends to stick to the palate or other portions of the mucous membrane in the oral cavity due to its nonporous smooth surface and extended decomposition. This is because the surface of polymer layers is dissolved into a sticky film.

However, since it is possible to easily control the diameter (several to tens of thousands of nanometers) of fibers in the web to be formed, the thickness (several to tens of thousands of micrometers) of the nano web, and the size of pores under modified conditions, the dissolvable nano web porous film of the invention can be formed in a variety of thickness as necessary. Accordingly, unlike the conventional nonporous film, the nonporous film of the invention is not only prevented from sticking to the surface of the oral cavity, but also rapidly delivers materials due to its wide specific surface area when applied to the mucous membrane of the oral cavity. Thus, the nonporous film has improved discharging effect to discharge medicines fast, and has excellent solubility and dissolvability.

Mode for the Invention

Hereinafter, the dissolvable nano web porous film according to the present invention will be described in detail with reference to the following Examples. It should be un derstocd that these examples is provided for illustrative purpose only, and do not limit the present invention.

### EXAMPLE 1

(1) Preparation of Polymer Solution for Forming Nano Web Porous Film

1.5 wt % avicel was ached to 70 wt % distilled water, and sufficiently uniformly dispersed at room temperature for about 10 minutes. Also, 0.6 wt % sucrose fatty acid ester was ailed to the solution, and completely dissolved therein. Then, 0.3 wt % sorbitol, 0.8 wt % propylene glycol, and 0.4 wt % glycerine were ailed to the resulting solution, and heated to 70° C., 23 wt % pullulan was slowly aided to the heated solution, and stirred to be completely dissolved in the solution. After heating the resulting solution to 80° C., 0.13 wt % carrageenan, 0.13 wt % locust bean gum (LBG), and 0.13 wt % xanthan gum were ailed to the heated resulting solution, and completely dispersed and dissolved. After cooling the resulting solution to 40° C., 0.46 wt % aspartame, 0.04 wt % acesulfame K, 0.3 wt % malic acid, 2 wt % menthol and 0.21 wt % citric acid were sequentially added thereto, followed by stirring them until they were uniformly dispersed and completely dissolved, thereby preparing a polymer solution. The solution had a viscosity of about 7,000˜8,000 mPa·s(25° C.). The solution was kept at room temperature for 6 to 24 hours to remove air bubbles.

(2) Formation of Nano Web Porous Film by Electrospinning

The polymer solution prepared at process (1) was stored in a syringe, and supplied to an inlet pipe by a syringe pump at a constant speed of 45 μl/h. The solution in a liquid phase was pushed out through a minute opening (an inner diameter of 0.2 mm) of a nozzle tip in a spinning unit via the inlet pipe. With an electric field of 15 kV applied, nano fibers were stacked and wound on the surface of a collector disposed at a pre-determined distance while the solvent is volatized or congealed, forming a nano web having a three-dimensional network structure.

(2)-1 Compression of the Nano Web Porous Film

The nano web of the three-dimensional network structure was compressed at a pressure of 2 atm to form a film having a two-dimensional plate structure. In this example, the nano web was compressed to forth a nano web porous film having a thickness of 10˜50 μm.

(3) Drying Nano Web Porous Film

The nano web porous film formed at process (2)-1 was dried by air heated to about 80˜110° C. for one minute to adjust its moisture content to 5˜20 wt %.

(4) Stabilization of Nano Web Porous Film

The nano web porous film dried at process (4) was kept in a thermo-hygrostat at a temperature of 25° C. and a relative humidity of 45% for 24 hours to be stabilized. Then, the nano web porous film had a moisture content of about 11-13 wt %.

The dissolvable nano web porous film was cut into pieces 2 cm wide by 3 cm long. With a piece as a sample, the nano web porous film of 10˜50 μm in thickness was evaluated with respect to its film forming properties and dissolvability. The film forming properties and dissolvability were evaluated by the following method.

(A) Evaluation of Film Forming Properties

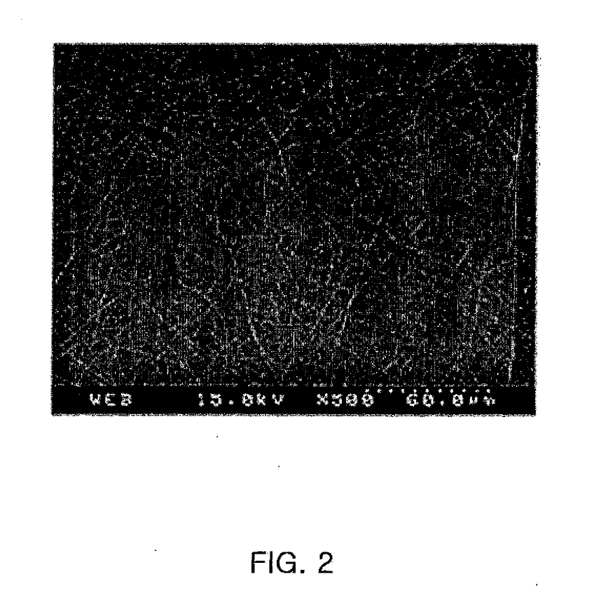

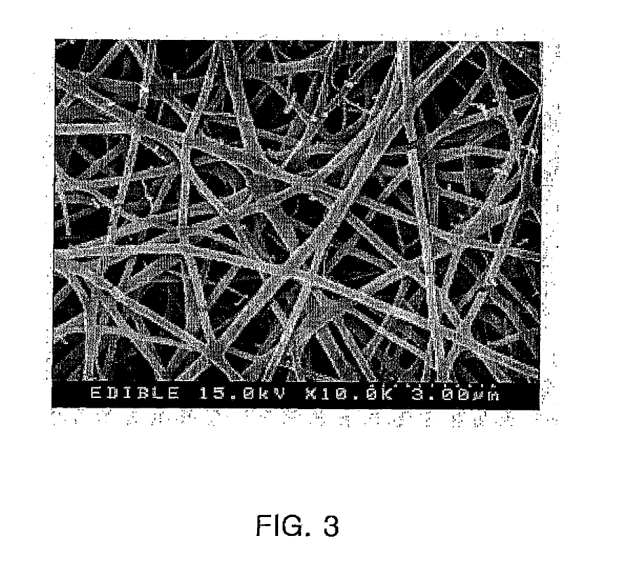

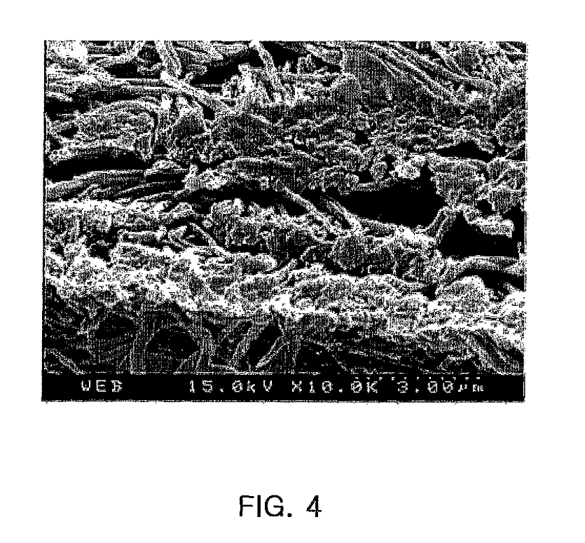

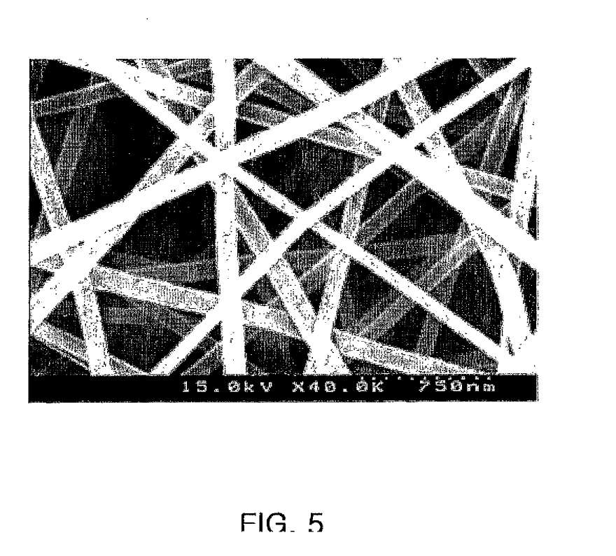

The sample film was examined using an electron microscope to obtain its forming state, size of the nano fiber, network structure, etc. Results are shown in FIGS. 2 to 5. The specific surface area of the dissolvable nano web porous film was measured by the BET (Brunauer, Emmett, Teller) method. In this example, the nano fiber had a diameter of about 200 nm, and the porous film had a specific surface area of 110 m²/g and porosity of 42%.

(B) Evaluation of Dissolvability

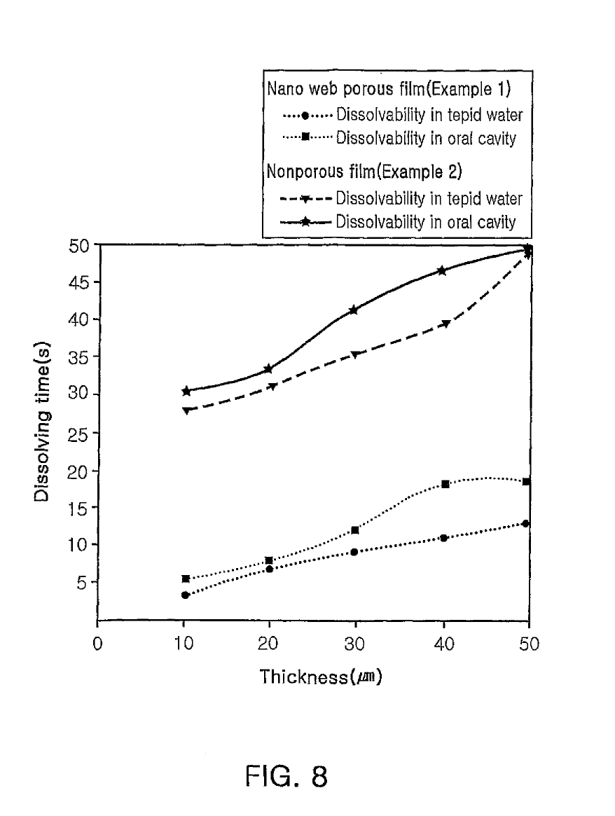

The sample film was put in tepid water at 35° C., and left after being shaken two or three times. Here, the container was carefully shaken such that the film was not stuck on the wall of the container. Then, the dissolvability of the film was evaluated by measuring time taken for complete dissolution of the film. The dissolvability of the film in the oral cavity was determined by putting the sample film in the mouth of an adult male and measuring time taken for complete dissolution of the film by saliva. Results of the dissolvability in tepid water and in the oral cavity are shown in FIG. 8. The porous film having a thickness of 20 μm this example was completely dissolved within 7 seconds in tepid water and within 8 seconds in the oral cavity.

### EXAMPLE 2

1.5 wt % avicel was added to 70 wt % distilled water, and sufficiently uniformly dispersed at room temperature for about 10 minutes. Also, 0.6 wt % sucrose fatty acid ester was ailed to the solution, and completely dissolved therein. Then, 0.3 wt % sorbitol, 0.8 wt % propylene glycol, and 0.4 wt % glycerine were aided to the resulting solution, and heated to 70° C. 23 wt % pullulan was slowly added to the heated solution, and stirred to be completely dissolved in the solution. After heating the resulting solution to 80° C., 0.1 wt % carrageenan, 0.1 wt % locust bean gum (LBG), and 0.1 wt % xanthan gum were added to the heated resulting solution, and completely dispersed and dissolved. After cooling the resulting solution to 40° C., 0.5 wt % aspartame, 0.1 wt % acesulfame K, 0.3 wt % malic acid, 2 wt % menthol, and 0.2 wt % citric acid were added thereto, followed by stirring them until they were uniformly dispersed and completely dissolved, thereby preparing a polymer solution.

The solution was kept for 6 hours or more to remove air bubbles. Then, the solution was coated to a thin film and cast to produce a film having a thickness in the range of 10˜50 μm. The resulting nonporous film was dried by heated air at about 60˜100° C. and kept in a thermo-hygrostat at a temperature of 25° C. and a relative humility of 45% for 24 hours to be stabilized. The nonporous film had a moisture content of about 11˜13 wt %. The nonporous film was cut into pieces 2 cm wile by 3 cm long.

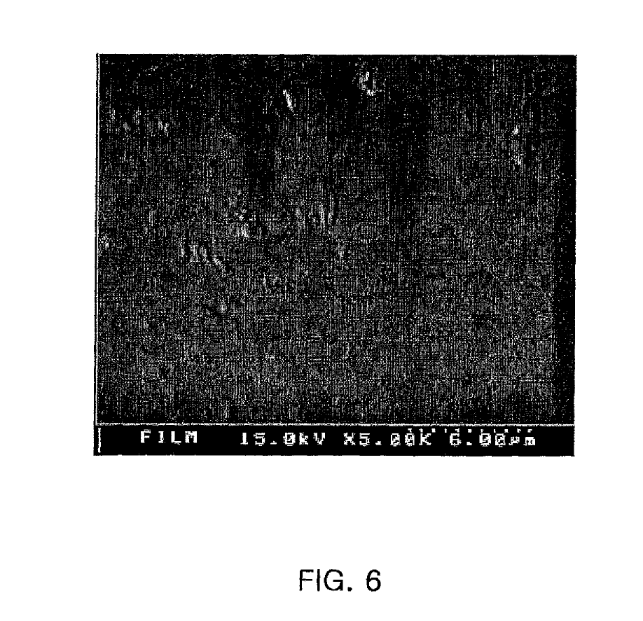

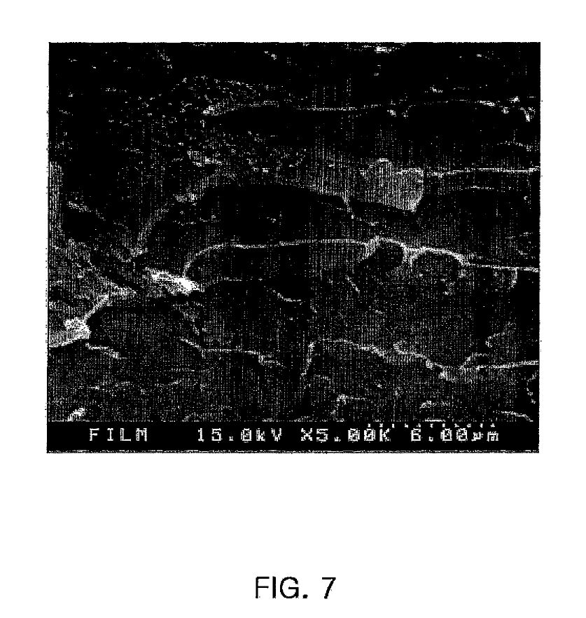

The nonporous film produced in this example was evaluated by the same method as in Example 1 with respect to film forming properties and dissolvability (evaluation of surface-active reaction of nonporous film). A film-forming state of the film was observed by an electron microscope, of which results are shown in FIGS. 6 and 7. Results of the dissolvability in tepid water and in the oral cavity are shown in FIG. 8. The nonporous film having a thickness of 20 μm was completely dissolved within 33 seconds in tepid water and within 35 seconds in the oral cavity.

### EXAMPLE 3

0.6 wt % sucrose fatty acid ester was ailed to 71.41 wt % distilled water, and sufficiently uniformly dispersed at room temperature. Then, 0.3 wt % sorbitol, 0.9 wt % propylene glycol and 0.4 wt % glycerine were added to the resulting solution, and heated to 70° C. 23 wt % hydroxypropylmethyl cellulose (HPMC) was slowly ailed to the heated solution, and stirred to be completely dissolved in the solution. After heating the resulting solution to 80° C., 0.1 wt % carrageenan, 0.1 wt % locust bean gum (LBG), and 0.1 wt % xanthan gum were ailed thereto, and completely dispersed and dissolved. After cooling the resulting solution to 40° C., 0.45 wt % aspartame, 0.04 wt % acesulfame K, 0.3 wt % malic acid, 2 wt % menthol and 0.3 wt % citric acid are added thereto, stirring to be uniformly dispersed were added thereto, followed by stirring them until they were uniformly dispersed and completely dissolved, preparing a polymer solution. The porous film was produced by the same method as in Example 1 except using the solution prepared in example 3, a nozzle tip having an inner diameter of 0.1˜10 mm, and electric field having a voltage of 10˜25 kV.

### EXAMPLE 4

After heating 77 wt % distilled water to 70° C., 21 wt % pectin, 0.5 wt % carrageenan, 0.5 wt % glycerine, 0.2 wt % sorbitol, and 0.8 wt % propylene glycol were added thereto, and stirred to be uniformly dispersed and completely dissolved, thereby producing a solution. Then, a porous film was formed of the resulting solution by the same method as in Example 3 except using the solution prepared in example 4.

### EXAMPLES 5 to 22

In Examples 5 to 22, films were produced using solutions formed of ingredients in Table 1 by the same methal as in Example 1. In Table 1, a quantity is expressed by wt % (% by weight).

TABLE 1

| | Examples | | | | | | | | | | | | | | | | | |
|---|---|---|---|---|---|---|---|---|---|---|---|---|---|---|---|---|---|---|
| Ingredient | 5 | 6 | 7 | 8 | 9 | 10 | 11 | 12 | 13 | 14 | 15 | 16 | 17 | 18 | 19 | 20 | 21 | 22 |
| Pectin | 21.7 | 21.6 | 21.6 | 21.6 | 21.6 | 23.1 | 0 | 0 | 0 | 0 | 0 | 0 | 0 | 0 | 0 | 0 | 0 | 0 |
| Pullulan | 0 | 0 | 0 | 0 | 0 | 0 | 21.6 | 21.6 | 21.6 | 21.6 | 21.6 | 23.1 | 0 | 0 | 0 | 0 | 0 | 0 |
| HPMC | 0 | 0 | 0 | 0 | 0 | 0 | 0 | 0 | 0 | 0 | 0 | 0 | 21.6 | 21.6 | 21.6 | 21.6 | 21.6 | 21.6 |
| Avicel | 1.5 | 1.5 | 1.5 | 1.5 | 1.5 | 0 | 1.5 | 1.5 | 1.5 | 1.5 | 1.5 | 0 | 1.5 | 1.5 | 1.5 | 1.5 | 1.5 | 0 |
| Sorbitol | 0.29 | 0.29 | 0.29 | 0.29 | 0.29 | 0.29 | 0.29 | 0.29 | 0.29 | 0.29 | 0.29 | 0.29 | 0.29 | 0.29 | 0.29 | 0.29 | 0.29 | 0.26 |
| Citric acid | 0.26 | 0.26 | 0.26 | 0.26 | 0.26 | 0.26 | 0.26 | 0.26 | 0.26 | 0.26 | 0.26 | 0.26 | 0.26 | 0.26 | 0.26 | 0.26 | 0.26 | 0.26 |
| Malic acid | 0.32 | 0.32 | 0.32 | 0.32 | 0.32 | 0.32 | 0.32 | 0.32 | 0.32 | 0.32 | 0.32 | 0.32 | 0.32 | 0.32 | 0.32 | 0.32 | 0.32 | 0.3 |
| Xanthan gum | 0 | 0.12 | 0.22 | 0 | 0.32 | 0 | 0 | 0.12 | 0.12 | 0 | 0.32 | 0 | 0 | 0.12 | 0.22 | 0 | 0.32 | 1.0 |
| LBG | 0.12 | 0 | 0.12 | 0 | 0 | 0.32 | 0.22 | 0 | 0.22 | 0 | 0 | 0.32 | 0.22 | 0 | 0.12 | 0 | 0 | 0.17 |
| Carrageenan | 0.12 | 0.22 | 0 | 0.2 | 0 | 0 | 0.12 | 0.22 | 0 | 0.2 | 0 | 0 | 0.12 | 0.1 | 0 | 0.2 | 0 | 0 |
| Guar gum | 0 | 0 | 0 | 0.12 | 0 | 0 | 0 | 0 | 0 | 0.12 | 0 | 0 | 0 | 0.12 | 0 | 0.12 | 0 | 0.7 |
| Acesulfame K | 0.03 | 0.03 | 0.03 | 0.03 | 0.03 | 0.03 | 0.03 | 0.03 | 0.03 | 0.03 | 0.03 | 0.03 | 0.03 | 0.03 | 0.03 | 0.03 | 0.03 | 0.03 |
| Aspartame | 0.4 | 0.4 | 0.4 | 0.42 | 0.42 | 0.42 | 0.4 | 0.4 | 0.4 | 0.42 | 0.42 | 0.42 | 0.4 | 0.4 | 0.4 | 0.42 | 0.42 | 0.42 |
| Glycerine | 0.4 | 0.4 | 0.4 | 0.4 | 0.4 | 0.4 | 0.4 | 0.4 | 0.4 | 0.4 | 0.4 | 0.4 | 0.4 | 0.4 | 0.4 | 0.4 | 0.4 | 0.4 |
| Propylene | 0.81 | 0.81 | 0.81 | 0.81 | 0.81 | 0.81 | 0.81 | 0.81 | 0.81 | 0.81 | 0.81 | 0.81 | 0.81 | 0.81 | 0.81 | 0.81 | 0.81 | 0.81 |
| glycol | | | | | | | | | | | | | | | | | | |
| Ethanol | 0.43 | 0.43 | 0.43 | 0.43 | 0.43 | 0.43 | 0.43 | 0.43 | 0.43 | 0.43 | 0.43 | 0.43 | 0.43 | 0.43 | 0.43 | 0.43 | 0.43 | 0.43 |
| DW | 73 | 73 | 73 | 73 | 73 | 73 | 73 | 73 | 73 | 73 | 73 | 73 | 73 | 73 | 73 | 73 | 73 | 73 |
| Sucrose | 0.62 | 0.62 | 0.62 | 0.62 | 0.62 | 0.62 | 0.62 | 0.62 | 0.62 | 0.62 | 0.62 | 0.62 | 0.62 | 0.62 | 0.62 | 0.62 | 0.62 | 0.62 |
| fatty acid | | | | | | | | | | | | | | | | | | |
| esters | | | | | | | | | | | | | | | | | | |
| SUM | 100 | 100 | 100 | 100 | 100 | 100 | 100 | 100 | 100 | 100 | 100 | 100 | 100 | 100 | 100 | 100 | 100 | 100 |

In Examples 5 to 22, solutions were prepared using ingredients and content shown in Table 1 by the following process. Avicel was ailed to distilled water (DW), and sufficiently uniformly dispersed at room temperature for about 10 minutes. Also, sucrose fatty acid ester was ailed to the solution, and completely dissolved therein. (In Examples 10, 16 and 22 where avicel was not used, sucrose fatty acid ester was added to distilled water, and sufficiently dissolved at room temperature for about 10 minutes.) Then, sorbitol, propylene glycol, and glycerine were added to the resulting solution and heated to 70° C. Pullulan, pectin, and hydroxypropylmethyl cellulose (HPMC) were slowly added to the heated solution, and stirred to be completely dissolved in the solution. After heating the resulting solution to 80° C., carrageenan, locust bean gum (LBG), xanthan gum and guar gum were added to the heated resulting solution, and completely dispersed and dissolved. After cooling the resulting solution to 40° C., aspartame, acesulfame K, ethanol, malic acid, and citric acid were sequentially added thereto, followed by stirring them until they were uniformly dispersed and completely dissolved, thereby preparing a polymer solution. A nano web porous film was produced using the solution by the same method as in Example 1.

### EXAMPLES 23 to 40

In Examples 23 to 40, films were produced using a solution formed of ingredients in

Table 2 by the same methal as in Example 1. In Table 2, a quantity is expressed by wt %.

TABLE 2

| | Example | | | | | | | | |
|---|---|---|---|---|---|---|---|---|---|
| Ingredient | 23 | 24 | 25 | 26 | 27 | 28 | 29 | 30 | 31 |
| Pectin | 21.6 | 21.6 | 21.6 | 21.6 | 21.6 | 21.6 | 0 | 0 | 0 |
| Pullulan | 0 | 0 | 0 | 0 | 0 | 0 | 21.6 | 21.6 | 21.6 |
| HPMC | 0 | 0 | 0 | 0 | 0 | 0 | 0 | 0 | 0 |
| Glycerine | 0 | 0 | 1.63 | 0 | 0 | 2.44 | 0 | 0 | 1.63 |
| Propylene | 1.81 | 0.8 | 0.81 | 0 | 2.44 | 0 | 1.81 | 0.8 | 0.81 |
| glycol | | | | | | | | | |
| Sorbitol | 0.63 | 1.64 | 0 | 2.44 | 0 | 0 | 0.63 | 1.64 | 0 |
| Acesulfame K | 0.03 | 0.03 | 0.03 | 0.03 | 0.03 | 0.03 | 0.03 | 0.03 | 0.03 |
| Citric acid | 0.26 | 0.26 | 0.26 | 0.26 | 0.26 | 0.26 | 0.26 | 0.26 | 0.26 |
| Malic acid | 0.32 | 0.32 | 0.32 | 0.32 | 0.32 | 0.32 | 0.32 | 0.32 | 0.32 |
| Avicel | 1.5 | 1.5 | 1.5 | 1.5 | 1.5 | 1.5 | 1.5 | 1.5 | 1.5 |
| LBG | 0.12 | 0.12 | 0.12 | 0.12 | 0.12 | 0.12 | 0.12 | 0.12 | 0.12 |
| Xanthan gum | 0.12 | 0.12 | 0.12 | 0.12 | 0.12 | 0.12 | 0.12 | 0.12 | 0.12 |
| Carrageenan | 0.12 | 0.12 | 0.12 | 0.12 | 0.12 | 0.12 | 0.12 | 0.12 | 0.12 |
| Aspartame | 0.44 | 0.44 | 0.44 | 0.44 | 0.44 | 0.44 | 0.44 | 0.44 | 0.44 |
| Ethanol | 0.43 | 0.43 | 0.43 | 0.43 | 0.43 | 0.43 | 0.43 | 0.43 | 0.43 |
| DW | 72 | 72 | 72 | 72 | 72 | 72 | 72 | 72 | 72 |
| Sucrose | 0.62 | 0.62 | 0.62 | 0.62 | 0.62 | 0.62 | 0.62 | 0.62 | 0.62 |
| fatty acid | | | | | | | | | |
| esters | | | | | | | | | |
| SUM | 100 | 100 | 100 | 100 | 100 | 100 | 100 | 100 | 100 |
| | Example | | | | | | | | |
| Ingredient | 32 | 33 | 34 | 35 | 36 | 37 | 38 | 39 | 40 |
| Pectin | 0 | 0 | 0 | 0 | 0 | 0 | 0 | 0 | 0 |
| Pullulan | 21.6 | 21.6 | 21.6 | 0 | 0 | 0 | 0 | 0 | 0 |
| HPMC | 0 | 0 | 0 | 21.6 | 21.6 | 21.6 | 21.6 | 21.6 | 21.6 |
| Glycerine | 0 | 0 | 2.44 | 0 | 0 | 1.63 | 0 | 0 | 2.44 |
| Propylene | 0 | 2.44 | 0 | 1.81 | 0.8 | 0.81 | 0 | 2.44 | 0 |
| glycol | | | | | | | | | |
| Sorbitol | 2.44 | 0 | 0 | 0.63 | 1.64 | 0 | 2.44 | 0 | 0 |
| Acesulfame K | 0.03 | 0.03 | 0.03 | 0.03 | 0.03 | 0.03 | 0.03 | 0.03 | 0.03 |
| Citric acid | 0.26 | 0.26 | 0.26 | 0.26 | 0.26 | 0.26 | 0.26 | 0.26 | 0.26 |
| Malic acid | 0.32 | 0.32 | 0.32 | 0.32 | 0.32 | 0.32 | 0.32 | 0.32 | 0.32 |
| Avicel | 1.5 | 1.5 | 1.5 | 1.5 | 1.5 | 1.5 | 1.5 | 1.5 | 1.5 |
| LBG | 0.12 | 0.12 | 0.12 | 0.12 | 0.12 | 0.12 | 0.12 | 0.12 | 0.12 |
| Xanthan | 0.12 | 0.12 | 0.12 | 0.12 | 0.12 | 0.12 | 0.12 | 0.12 | 0.12 |
| gum | | | | | | | | | |
| Carrageenan | 0.12 | 0.12 | 0.12 | 0.12 | 0.12 | 0.12 | 0.12 | 0.12 | 0.12 |
| Aspartame | 0.44 | 0.44 | 0.44 | 0.44 | 0.44 | 0.44 | 0.44 | 0.44 | 0.44 |
| Ethanol | 0.43 | 0.43 | 0.43 | 0.43 | 0.43 | 0.43 | 0.43 | 0.43 | 0.43 |
| DW | 72 | 72 | 72 | 72 | 72 | 72 | 72 | 72 | 72 |
| Sucrose fatty | 0.62 | 0.62 | 0.62 | 0.62 | 0.62 | 0.62 | 0.62 | 0.62 | 0.62 |
| acid esters | | | | | | | | | |
| SUM | 100 | 100 | 100 | 100 | 100 | 100 | 100 | 100 | 100 |

In Examples 23 to 40, solutions were prepared using ingredients in Table 2 by the following process. Avicel was added to distilled water (DW), and sufficiently uniformly dispersed at room temperature for about 10 minutes. Also, sucrose fatty acid ester was ailed to the solution, and completely dissolved therein. Then, sorbitol, propylene glycol, and glycerine were aided to the resulting solution and heated to 70° C. Pullulan, pectin, and hydroxypropylmethyl cellulose (HPMC) were slowly ailed to the heated solution, and stirred to be completely dissolved in the solution. After heating the resulting solution to 80° C., carrageenan, locust bean gum (LBG) and xanthan gum were ailed to the heated resulting solution, and completely dispersed and dissolved. After cooling the resulting solution to 40° C., aspartame, acesulfame K, ethanol, malic acid, and citric acid were sequentially added thereto, followed by stirring them until they were uniformly dispersed and completely dissolved, thereby preparing a polymer solution. A nano web porous film was produced using the solution by the same method as in Example 1.

### EXAMPLES 41 to 48

In Examples 41 to 48, films were formed using solutions formed of ingredients (medical supplies) in Table 3 by the same method as in Example 1. In Table 3, a quantity is expressed by wt %.

TABLE 3

| | Example | | | | | | | |
|---|---|---|---|---|---|---|---|---|
| Ingredient | 41 | 42 | 43 | 44 | 45 | 46 | 47 | 48 |
| Pullulan | 21.6 | 21.6 | 21.6 | 21.6 | 21.6 | 21.6 | 21.6 | 21.6 |
| Glycerine | 0.4 | 0.4 | 0.4 | 0.4 | 0.4 | 0.4 | 0.4 | 0.4 |
| Propylene glycol | 0.8 | 0.8 | 0.8 | 0.8 | 0.8 | 0.8 | 0.8 | 0.8 |
| Acesulfame K | 0.03 | 0.03 | 0.03 | 0.03 | 0.03 | 0.03 | 0.03 | 0.03 |
| Citric acid | 0.26 | 0.26 | 0.26 | 0.26 | 0.26 | 0.26 | 0.26 | 0.26 |
| Malic acid | 0.32 | 0.32 | 0.32 | 0.32 | 0.32 | 0.32 | 0.32 | 0.32 |
| Dextromethorphan HBr | 2.0 | 0 | 0 | 0 | 0 | 0 | 0 | 0 |
| Phenylephrine HCl | 0 | 2.0 | 0 | 0 | 0 | 0 | 0 | 0 |
| Alkylamines chloropheniramine | 0 | 0 | 2.0 | 0 | 0 | 0 | 0 | 0 |
| Loperamide HCl | 0 | 0 | 0 | 2.0 | 0 | 0 | 0 | 0 |
| Nicotine | 0 | 0 | 0 | 0 | 2.0 | 0 | 0 | 0 |
| Magnesium Stearate | 0 | 0 | 0 | 0 | 0 | 2.0 | 0 | 0 |
| Oxybutynin | 0 | 0 | 0 | 0 | 0 | 0 | 2.0 | 0 |
| Estradiol | 0 | 0 | 0 | 0 | 0 | 0 | 0 | 2.0 |
| LBG | 0.12 | 0.12 | 0.12 | 0.12 | 0.12 | 0.12 | 0.12 | 0.12 |
| Xanthan gum | 0.12 | 0.12 | 0.12 | 0.12 | 0.12 | 0.12 | 0.12 | 0.12 |
| Carrageenan | 0.12 | 0.12 | 0.12 | 0.12 | 0.12 | 0.12 | 0.12 | 0.12 |
| Aspartame | 0.4 | 0.4 | 0.4 | 0.4 | 0.4 | 0.4 | 0.4 | 0.4 |
| DW | 72 | 72 | 72 | 72 | 72 | 72 | 72 | 72 |
| Sucrose fatty acid esters | 0.63 | 0.63 | 0.63 | 0.63 | 0.63 | 0.63 | 0.63 | 0.63 |
| Menthol | 1.2 | 1.2 | 1.2 | 1.2 | 1.2 | 1.2 | 1.2 | 1.2 |
| Sum | 100 | 100 | 100 | 100 | 100 | 100 | 100 | 100 |

In Examples 41 to 48, solutions were prepared using ingredients shown in Table 3 by the following process. Sucrose fatty acid ester was ailed to distilled water heated to 50° C., and completely dissolved. Then, propylene glycol and glycerine were ailed to the resulting solution, and slowly heated to 70° C. Pullulan was slowly ailed to the heated solution, stirred to be completely dissolved in the solution. After heating the resulting solution to 80° C., carrageenan, locust bean gum (LBG), and xanthan gum were ailed to the heated resulting solution, and completely dispersed and dissolved. After cooling the resulting solution to 40° C., aspartame, acesulfame K, malic acid, menthol, and citric acid were sequentially added thereto. Then, a pharmaceutical active agent (e.g., dextromethorphan HBr in Example 41, phenylephrine HCl in Example 42, alkylamines chloropheniramine in Example 43, loperamide HCl in Example 44, Nicotine for Example 45, magnesium stearate in Example 46, oxybutynin in Example 47, and extradiol in Example 48) was added to the solution. The solution was stirred until all ingredients were uniformly dispersed and completely dissolved, thereby preparing a polymer solution. A nano web porous film was produced using the solution by the same method as in Example 1, and cut in pieces of a suitable size capable of ensuring a suitable pharmaceutical content, thereby preparing a pharmaceutical nano web porous film.

### EXAMPLES 49 to 66

In Examples 49 to 66, films were produced using solutions (food) formed of ingredients shown in Table 4 by the same method as in Example 1. In Table 4, a quantity is expressed by wt %.

TABLE 4

| | Example | | | | | | | | |
|---|---|---|---|---|---|---|---|---|---|
| Ingredient | 49 | 50 | 51 | 52 | 53 | 54 | 55 | 56 | 57 |
| Pectin | 21.3 | 21.3 | 21.3 | 21.3 | 21.3 | 21.3 | 0 | 0 | 0 |
| Pullulan | 0 | 0 | 0 | 0 | 0 | 0 | 21.3 | 21.3 | 21.3 |
| HPMC | 0 | 0 | 0 | 0 | 0 | 0 | 0 | 0 | 0 |
| Glycerine | 0.35 | 0.35 | 0.35 | 0.35 | 0.35 | 0.35 | 0.35 | 0.35 | 0.35 |
| Propylene glycol | 0.76 | 0.76 | 0.76 | 0.76 | 0.76 | 0.76 | 0.76 | 0.76 | 0.76 |
| Sorbitol | 0.18 | 0.18 | 0.18 | 0.18 | 0.18 | 0.18 | 0.18 | 0.18 | 0.18 |
| Acesulfame K | 0.03 | 0.03 | 0.03 | 0.03 | 0.03 | 0.03 | 0.03 | 0.03 | 0.03 |
| Citric acid | 0.26 | 0.26 | 0.26 | 0.26 | 0.26 | 0.26 | 0.26 | 0.26 | 0.26 |
| Malic acid | 0.32 | 0.32 | 0.32 | 0.32 | 0.32 | 0.32 | 0.32 | 0.32 | 0.32 |
| Avicel | 1.5 | 1.5 | 1.5 | 1.5 | 1.5 | 1.5 | 1.5 | 1.5 | 1.5 |
| LBG | 0.12 | 0.12 | 0.12 | 0.12 | 0.12 | 0.12 | 0.12 | 0.12 | 0.12 |
| Xanthan gum | 0.12 | 0.12 | 0.12 | 0.12 | 0.12 | 0.12 | 0.12 | 0.12 | 0.12 |
| Carrageenan | 0.12 | 0.12 | 0.12 | 0.12 | 0.12 | 0.12 | 0.12 | 0.12 | 0.12 |
| Aspartame | 0.43 | 0.43 | 0.43 | 0.43 | 0.43 | 0.43 | 0.43 | 0.43 | 0.43 |
| Ethanol | 0.43 | 0.43 | 0.43 | 0.43 | 0.43 | 0.43 | 0.43 | 0.43 | 0.43 |
| DW | 68 | 68 | 68 | 68 | 68 | 68 | 68 | 68 | 68 |
| Sucrose fatty | 0.62 | 0.62 | 0.62 | 0.62 | 0.62 | 0.62 | 0.62 | 0.62 | 0.62 |
| acid esters | | | | | | | | | |
| Polysorbate 80 | 0 | 0 | 0 | 0 | 0 | 0 | 0 | 0 | 0 |
| Menthol | 1.88 | 1.88 | 1.88 | 1.88 | 1.88 | 1.88 | 1.88 | 1.88 | 1.88 |
| Mint flavored | 0.3 | 0.67 | 0.37 | 0.27 | 0.47 | 0.27 | 0.3 | 0.67 | 0.37 |
| Powder | | | | | | | | | |
| Lemon flavor | 2.94 | 0 | 0 | 0 | 0 | 0 | 2.94 | 0 | 0 |
| Citrus spice | 0.34 | 0.54 | 0.34 | 0.45 | 0.25 | 0.32 | 0.34 | 0.54 | 0.34 |
| mint flavor | | | | | | | | | |
| Spearmint | 0 | 2.37 | 0 | 0 | 0 | 0 | 0 | 2.37 | 0 |
| flavor | | | | | | | | | |
| Coffee flavor | 0 | 0 | 0 | 0 | 0 | 0 | 0 | 0 | 0 |
| Peach flavor | 0 | 0 | 2.87 | 0 | 0 | 0 | 0 | 0 | 2.87 |
| Natural orange | 0 | 0 | 0 | 2.86 | 0 | 0 | 0 | 0 | 0 |
| oil flavor | | | | | | | | | |
| Cherry flavor | 0 | 0 | 0 | 0 | 2.86 | 0 | 0 | 0 | 0 |
| Strawberry | 0 | 0 | 0 | 0 | 0 | 2.99 | 0 | 0 | 0 |
| flavor | | | | | | | | | |
| SUM | 100 | 100 | 100 | 100 | 100 | 100 | 100 | 100 | 100 |
| | Example | | | | | | | | |
| Ingredient | 58 | 59 | 60 | 61 | 62 | 63 | 64 | 65 | 66 |
| Pectin | 0 | 0 | 0 | 0 | 0 | 0 | 0 | 0 | 0 |
| Pullulan | 21.3 | 21.3 | 21.3 | 0 | 0 | 0 | 0 | 0 | 0 |
| HPMC | 0 | 0 | 0 | 21.3 | 21.3 | 21.3 | 21.3 | 21.3 | 21.3 |
| Glycerine | 0.35 | 0.35 | 0.35 | 0.35 | 0.35 | 0.35 | 0.35 | 0.35 | 0.35 |
| Propylene glycol | 0.76 | 0.76 | 0.76 | 0.76 | 0.76 | 0.76 | 0.76 | 0.76 | 0.76 |
| Sorbitol | 0.18 | 0.18 | 0.18 | 0.18 | 0.18 | 0.18 | 0.18 | 0.18 | 0.18 |
| Acesulfame K | 0.03 | 0.03 | 0.03 | 0.03 | 0.03 | 0.03 | 0.03 | 0.03 | 0.03 |
| Citric acid | 0.26 | 0.26 | 0.26 | 0.26 | 0.26 | 0.26 | 0.26 | 0.26 | 0.26 |
| Malic acid | 0.32 | 0.32 | 0.32 | 0.32 | 0.32 | 0.32 | 0.32 | 0.32 | 0.32 |
| Avicel | 1.5 | 1.5 | 1.5 | 1.5 | 1.5 | 1.5 | 1.5 | 1.5 | 1.5 |
| LBG | 0.12 | 0.12 | 0.12 | 0.12 | 0.12 | 0.12 | 0.12 | 0.12 | 0.12 |
| Xanthan gum | 0.12 | 0.12 | 0.12 | 0.12 | 0.12 | 0.12 | 0.12 | 0.12 | 0.12 |
| Carrageenan | 0.12 | 0.12 | 0.12 | 0.12 | 0.12 | 0.12 | 0.12 | 0.12 | 0.12 |
| Aspartame | 0.43 | 0.43 | 0.43 | 0.43 | 0.43 | 0.43 | 0.43 | 0.43 | 0.43 |
| Ethanol | 0.43 | 0.43 | 0.43 | 0.43 | 0.43 | 0.43 | 0.43 | 0.43 | 0.43 |
| DW | 68 | 68 | 68 | 68 | 68 | 68 | 68 | 68 | 68 |
| Sucrose fatty | 0 | 0 | 0 | 0 | 0 | 0 | 0 | 0 | 0 |
| acid esters | | | | | | | | | |
| Polysorbate 80 | 0.62 | 0.62 | 0.62 | 0.62 | 0.62 | 0.62 | 0.62 | 0.62 | 0.62 |
| Menthol | 1.88 | 1.88 | 1.88 | 1.88 | 1.88 | 1.88 | 1.88 | 1.88 | 1.88 |
| Mint flavored | 0.27 | 0.47 | 0.27 | 0.3 | 0.67 | 0.37 | 0.27 | 0.47 | 0.27 |
| Powder | | | | | | | | | |
| Lemon flavor | 0 | 0 | 0 | 2.94 | 0 | 0 | 0 | 0 | 0 |
| Citrus spice | 0.45 | 0.25 | 0.32 | 0.34 | 0.54 | 0.34 | 0.45 | 0.25 | 0.32 |
| mint flavor | | | | | | | | | |
| Spearmint | 0 | 0 | 0 | 0 | 2.37 | 0 | 0 | 0 | 0 |
| flavor | | | | | | | | | |
| Coffee flavor | 0 | 0 | 0 | 0 | 0 | 0 | 0 | 0 | 0 |
| Peach flavor | 0 | 0 | 0 | 0 | 0 | 2.87 | 0 | 0 | 0 |
| Natural orange | 2.86 | 0 | 0 | 0 | 0 | 0 | 2.86 | 0 | 0 |
| oil flavor | | | | | | | | | |
| Cherry flavor | 0 | 2.86 | 0 | 0 | 0 | 0 | 0 | 2.86 | 0 |
| Strawberry | 0 | 0 | 2.99 | 0 | 0 | 0 | 0 | 0 | 2.99 |
| flavor | | | | | | | | | |
| SUM | 100 | 100 | 100 | 100 | 100 | 100 | 100 | 100 | 100 |

In Examples 49 to 66, solutions were prepared using ingredients in Table 4 by the following process. Avicel was ailed to distilled water (DW), and sufficiently uniformly dispersed at room temperature for about 10 minutes. Also, sucrose fatty acid ester and polysorbate 80 were added to the solution, and completely dissolved therein. Then, sorbitol, propylene glycol, and glycerine were added to the resulting solution and heated to 70° C. Pullulan, pectin and hydroxypropylmethyl cellulose (HPMC) were slowly added to the heated solution, and stirred to be completely dissolved in the solution. After heating the resulting solution to 80° C., carrageenan, locust bean gum (LBG) and xanthan gum were added to the heated resulting solution, and completely dispersed and dissolved. After cooling the resulting solution to 40° C., aspartame, acesulfame K, ethanol, malic acid, citric acid, menthol, a mint flavored powder, a lemon flavor, a citrus spice mint flavor, a spearmint flavor, a coffee flavor, a peach flavor, a natural orange oil flavor, a cheery flavor, and a strawberry flavor were sequentially added thereto, followed by stirring them until they were uniformly dispersed and completely dissolved, thereby preparing a polymer solution. A nano web porous film was produced using the solution by the same method as in Example 1.

### EXAMPLES 67 to 72

In Examples 67 to 72, films were formed using solutions formed of ingredients (for oral cleansing) in Table 5 by the same method as in Example 1. In Table 5, a quantity is expressed by wt %.

TABLE 5

| | Example | | | | | |
|---|---|---|---|---|---|---|
| Ingredient | 67 | 68 | 69 | 70 | 71 | 72 |
| Pullulan | 21.0 | 21.0 | 21.0 | 21.0 | 21.0 | 21.0 |
| Glycerine | 0.29 | 0.29 | 0.29 | 0.29 | 0.29 | 0.29 |
| Propylene glycol | 0.62 | 0.62 | 0.62 | 0.62 | 0.62 | 0.62 |
| Sorbitol | 0.19 | 0.19 | 0.19 | 0.19 | 0.19 | 0.19 |
| Acesulfame K | 0.03 | 0.03 | 0.03 | 0.03 | 0.03 | 0.03 |
| Citric acid | 0.26 | 0.26 | 0.26 | 0.26 | 0.26 | 0.26 |
| Malic acid | 0.32 | 0.32 | 0.32 | 0.32 | 0.32 | 0.32 |
| Avicel | 1.2 | 1.2 | 1.2 | 1.2 | 1.2 | 1.2 |
| LBG | 0.12 | 0.12 | 0.12 | 0.12 | 0.12 | 0.12 |
| Xanthan gum | 0.12 | 0.12 | 0.12 | 0.12 | 0.12 | 0.12 |
| Carrageenan | 0.12 | 0.12 | 0.12 | 0.12 | 0.12 | 0.12 |
| Aspartame | 0.33 | 0.33 | 0.33 | 0.33 | 0.33 | 0.33 |
| Ethanol | 0.12 | 0.12 | 0.12 | 0.12 | 0.12 | 0.12 |
| DW | 71 | 71 | 71 | 71 | 71 | 71 |
| Sucrose fatty | 0.62 | 0.62 | 0.62 | 0 | 0 | 0 |
| acid esters | | | | | | |
| Polysorbate 80 | 0 | 0 | 0 | 0.62 | 0.62 | 0.62 |
| Menthol | 1.35 | 1.35 | 1.35 | 1.35 | 1.35 | 1.35 |
| Eucapyptus oil | 0.5 | 0.5 | 0.5 | 0.5 | 0.5 | 0.5 |
| Cetylpyridinium | 0.3 | 0.3 | 0.3 | 0.3 | 0.3 | 0.3 |
| Chloride | | | | | | |
| Methyl salicylate | 0.2 | 0.2 | 0.2 | 0.2 | 0.2 | 0.2 |
| Mint flavored | 0.57 | 0.57 | 0.3 | 0.57 | 0.57 | 0.3 |
| powder | | | | | | |
| Spearmint flavor | 0.3 | 0 | 0.22 | 0.3 | 0 | 0.22 |
| Citrus spice | 0.44 | 0.44 | 0.44 | 0.44 | 0.44 | 0.44 |
| mint flavor | | | | | | |
| Peach flavor | 0 | 0.3 | 0.35 | 0 | 0.3 | 0.35 |
| SUM | 100 | 100 | 100 | 100 | 100 | 100 |

In Examples 67 to 72, solutions were prepared using ingredients shown in Table 5 by the following process. Avicel was ailed to distilled water (DW), and sufficiently uniformly dispersed at room temperature for about 10 minutes. Also, sucrose fatty acid ester and polysorbate 80 were added to the solution, and completely dissolved therein. Then, sorbitol, propylene glycol, and glycerine were ailed to the resulting solution and heated to 70° C. Pullulan was slowly ailed to the heated solution, and stirred to be completely dissolved in the solution. After heating the resulting solution to 80° C., carrageenan, locust bean gum (LBG), xanthan gum and guar gum are added thereto, and completely dispersed and dissolved. After cooling the resulting solution to 40° C., aspartame, acesulfame K, ethanol, malic acid, citric acid, menthol, eucalyptus oil, cetylpyridinium chloride, methyl salicylate, a mint flavored powder, a citrus spice mint flavor, a spearmint flavor, and a peach flavor were ailed thereto, followed by stirring them until they were uniformly dispersed and completely dissolved, thereby preparing a polymer solution. A nano web porous film was produced using the solution by the same methal as in Example 1.

While the present invention has been shown and described in connection with the preferred embodiments, it will be apparent to those skilled in the art that modifications and variations can be made without departing from the spirit and scope of the invention as defined by the appended claims.

1. A dissolvable nano web porous film, wherein the nano web porous film has a network structure formed by stacking nano fibers, the nano fibers having a thickness of 10-50,000 nm and formed of a composition comprising a nano fiber forming polymer, a gelling agent, and a plasticizer.

2. The porous film according to claim 1, wherein the composition further comprises at least one of an active component and an additive.

3. The porous film according to claim 1, wherein the nano fiber forming polymer has an average molecular weight of 1,000-2,000,000 g/mol.

4. The porous film according to claim 1, wherein the nano fiber-forming polymer is at least one material selected from the group consisting of pullulan, hydroxypropylmethyl cellulose (HPMC), hydroxymethyl cellulose (HMC), hydroxyethyl cellulose (HEC), hydroxypropyl cellulose (HPC), ethyl cellulose (EC), methyl cellulose (MC), hydroxypropylmethyl cellulose phthalate (HPMCP), hydroxypropylmethyl cellulose acetate succinate (HPMCAS), cellulose acetate phthalate (CAP), carboxymethyl cellulose (CMC), polyvinyl pyrrolidone (PVP), polyvinyl alcohol (PVA), sodium alginate, polyethylene glycol, locust bean gum, xanthan gum, tragacanth gum, guar gum, acacia gum, arabic gain, polyacrylic acid, methyl methacrylate copolymer, amylose, dextrin, chitin, chitosan, levan, elsinan, collagen, gelatin, glucan, high amylose starch (corn starch, potato starch, wheat starch, rice starch, mung-bean starch, yellow bean starch, etc.), ester derivatives of starch, soy protein isolate, whey protein isolate, casein, gellan and pectin, and is added in an amount of 1˜99 wt % to a total amount of the composition.

5. The porous film according to claim 1, wherein the gelling agent is at least one material selected from the group consisting of locust bean gum, xanthan gum, carrageenan, gelatin, gellan gum, agar, alginates, guar gum, starch, pectin, ethyl cellulose, methyl cellulose, sodium carboxymethyl cellulose and polyethylene glycol, and is added in an amount of 0.1˜50 wt % to 100 wt % nano fiber forming polymer.

6. The porous film according to claim 1, wherein the plasticizer is at least one material selected from the group consisting of sorbitol, mannitol, glycerin, propylene glycol, polyethylene glycol, fatty acid glycerol monoester, sugar alcohol, monosaccharide, oligosaccharide, triethylene glycol, myvacet, triethyl citrate, triacetin, propylene glycol monocaprylate, propylene glycol dicaprylate and miglyol, all being organic compounds of polyol containing at least one hydroxyl group, and is added in an amount of 0.1˜40 wt % to 100 wt % nano fiber forming polymer.

7. The porous film according to claim 2, wherein the active component is at least one selected from the group consisting of: α-adrenergic agonists; β-adrenergic agonists; α-adrenergic blocking agents; β-adrenergic blocking agents; alcohol prohibition agents; aldose-reductase inhibitors; anabolic agents; narcotic analgesics; derivatives of morphine; non-narcotic analgesics, preferably, salicylates and derivatives thereof; androgens; anesthetics; appetite inhibitors; anthelmintics (taenia, nematode, filarial,schistosome, and fluke eliminating active agents); antiacne agents; antiallergic agents; antiamebic drugs (ameba eliminating agents); antiandrogens; angina therapeutic agents; antiarrhythmic drugs; antiarteriosclerotic agents; antiarthritic and antirheumatic agents; antimicrobial agents (antibiotics), aminoglycosides, amphenicols, ansamycins, β-lactams (particularly, carbapenems, cephalosporins, cephamycins, monolactams, oxacephems, and penicillins), lincosamides, macrolides, polypeptides, and tetracyclines; synthetic antimicrobials, 2,4-diaminopyrimidines, nitrofurans, quinolones and quinolone analogues, sulfonamides, and sulfones; anticholinergics; anticonvulsants; antidepressants, bicyclic antidepressants, hydrazides, hydrazines, pyrrolidones, tetracycline antidepressants; tricyclic antidepressants, polycyclic imides; antidiabetic agents, biguanides, sulfonylurea derivatives; antidiarrheals; antidiuretic agents; antiestrogens; antifungal agents/fungicides, polyenes; synthetic antifungal agent/fungicide, allyl amines, imidazoles, triazoles; antiglaucomatous agents; antigonado-trophins; antigout agents; antihistamines, alkylamine derivatives, atninoalkyl ethers, ethylenediamine derivatives, piperazines, tricyclic compounds (particularly, phenothiazine); anti-hyperlipoproteinemia agents (fat inhibitors), aryloxy alkane acid derivatives (particularly, clofibric acid derivatives and analogues), bile acid-masking materials, HMG-CoA-reductase inhibitors, nicotinic acid derivatives, thyroid hormones and analogues thereof; antihypertensive drugs/hypotensives, benzothiadiazine derivatives, N-carboxylalkyl-(peptile/lactam) derivatives, guanidine derivatives, hydrazines/phthalazins, imidazole derivatives, quaternary ammonium compounds, quinazoline derivatives, reserpine derivatives, sulfonamide derivatives; antihyperthyroid agents; antihypotensive agents; antihypothyroid agents; non-steroidal antiinflam mato ry drugs (antiphlogistics), aminoarylcaxboxyl acid, arylacetic acid, arylbutyric acid, aryicaboxylic acid, arylpropionic acid derivatives, pyrazoles, pyrazolones, salicylic acid derivatives, thiazine carboxamide; antimalarial drugs, quinines, salts thereof, acids thereof and derivatives thereof; antimigraine drugs; retching inhibitors; antineoplastic drugs, alkylating drugs (particularly, alkyl sulfonates, aziridines, ethylenimines, methyl melamines, nitrogen mustard gases, nitrosoureas); antibiotics, antimetabolic agents (particularly, folinic acid analogues, purine analogues, pyrimidine analogues), enzymes, interferons, interleukins; hormonal antineoplastics, androgens, antiadrenalines, antiandrogens, antiestrogens (particularly, aromatase inhibitors); antineoplastic food additives; antiparkinsonian agents; antipheochrornocytoma agents; antipneumocystis agents; prostatic hypertrophy therapeutic agents; protozoacides, preferably, antileishmania, antitrichomonal and anti-trypanosoma drugs; antiprurignous agents; antipsoriasis drugs; antineuroleptic agents, butyrophenones, phenothiazines, thioxanthenes, tricyclic drugs, 4-arylpiperazines, 4-arylpiperidines; antipyretics; antirickettsia agents; antiseborrheic agents; antiseptics, guanidines, halogens and halogen compounds, nitrofurans, phenols, quinolines; anticonvulsants/convulsion inhibitors; antithrombotics; antitussives; antiulcer drugs; uric acid eliminating agents (antiurolithiasis agents); antiophidicas; antiviral drugs, purines, pyrimidinones; anxiolytic, arylpiperazines, benzodiazepine derivatives, carbamates; benzodiazepine antagonists; bronchodilators, ephedrine derivatives, quaternary animoniuin compounds, xanthine derivatives; calcium channel blockers, arylalkylamines, dihydropyridine derivatives, piperazine derivatives; calcium modulators; cardiac stimulants; chelate or complex forming agents; cholecystokinin antagonists; antigallstone agents; bile secretagogues; parasympathomimetic drugs; cholinesterase inhibitors; cholinesterase rehabilitation materials; CNS stimulants; decongestants; tooth decay prevention agents; decolorants; diuretics, organomercury compounds, pteridines, purines, steroids, sulfonamides, uracils; dopamine receptor agonists; ectoparasite eliminating agents; enzymes, digestive enzymes, penicillin inactivating enzymes, proteolytic enzymes; enzyme inducers; steroidal and non-steroidal estrogens; gastric acid secretion inhibitors; glucocorticoids; gonadotropic activators; gonadotropins; growth hormone inhibitors; growth hormone releasing factors; growth stimulants; anticoagulants; heparin antagonists; liver protective agents; liver disease therapeutic agents; immune modulators; immune inhibitors; ion exchange resins; prolactins; LH-RH agonists; lipotropic agents; lupus erythematosus eliminating agents; mineralocorticoids; miotic agents; monoamine oxidase inhibitors; mucolytic agents; muscle relaxants; narcotic antagonists; neuroprotective agents; nootropics; ophthalmic agents; ovarian hormones; oxytocic agents; pepsin inhibitors; peristaltic stimulants; progestogens; prolactin inhibitors; prostaglandins and analogues thereof; protease inhibitors; respiratory stimulants; hardening agents; sedatives/somnifacients, non-cyclic ureides, alcohols, amides, barbiturates acid derivatives, benzodiazepine derivatives, bromides, carbamates, chloral derivatives, piperidinediones, quinazolone derivatives; thrombolytic agents; thyroid hormones;
uricosuric agents; vasodilators (cerebrum); vasodilators (coronary arteries); vasodilators (peripheral nerve); blood vessel protective agents; vitamins, provitamins, vitamin extracts, vitamin derivatives; and trauma therapeutic agents.

8. The porous film according to claim 7, wherein the active component is added in an amount of 0.01˜40 wt % in the final nano web porous film.

9. The porous film according to claim 2, wherein the additive is at least one selected from the group consisting of: saccharides such as white sucrose, maltose, fructose, galactooligosaccharide, galactose, fractooligosaccharide, dextrin, ion starch syrup, malt syrup, lactose, glucose, sorbitol, mannitol, xylitol, inositol, etc.; saliva stimulating agents such as citric, lactic, maleic, succinic, ascorbic, adipic, fumaric and tartaric acids; sweeteners such as monosaccharide, disaccharide, ribose, glucose, mannose, galactose, fructose, sucrose, maltose, invert sugars, corn syrup solids, glythyl retinoate, white sugars, water soluble artificial sweeteners; thickening agents such as methyl cellulose, carboxyl methyl cellulose, etc.; masking agents such as citric, tartaric and fumaric acids; dyes; coloring agents such as natural pigments, tar pigments, etc.; refreshing agents such as menthol, peppermint oil, peppermint, spearmint, etc.; flavoring agents such as natural or synthetic fragrances; and nutrient, plant extracts, herbal components, vitamins, minerals, nitroglycerin, catechin, polyphenols, enzymes, emulsifier, seasonings, fragrances, fats and oil, adenosine, coenzyme Q10, vitamin E, vitamin C, fibroin, amyloglucosidase, albutin, hyaluronic acid, D/DL-pantheilol, neomycin, and hydrocortisone acetate.

10. The porous film according to claim 9, wherein the additive is added in an amount of 0.01˜40 wt % in the final nano web porous film.

11. The porous film according to claim 1, wherein the porous film has porosity of 10˜90%.

12. The porous film according to claim 1, wherein the porous film has a specific surface area of 0.1˜1,000 m²/g.

13. The porous film according to claim 1, wherein the porous film has a thickness of 5-500 μm.

14. A method of preparing a dissolvable nano web porous film, comprising:
producing a polymer melt or a polymer solution of a composition comprising a nano fiber forming polymer, a gelling agent and a plasticizer, as essential components and an active component, and additives, as optional components; spinning the polymer melt or solution to form a nano web porous film having nano webs stacked in a network structure;
drying the nano web porous film; and
stabilizing the nano web porous film.

15. The method according to claim 14, wherein the spinning step is performed by one method selected from the group consisting of electro spinning, spun-lace, spun-bond, melt-blown, and flash spinning.

16. The method according to claim 14, further comprising compressing the nano web porous film after the spinning.
